(19) United States
(12) Patent Application Publication
(10) Pub. No.: US 2023/0055404 A1
(43) Pub. Date: Feb. 23, 2023

(54) SOLID PREPARATION, AND PREPARATION METHOD THEREFOR AND USE THEREOF

(71) Applicant: Sinotherapeutics Inc., Shanghai (CN)

(72) Inventors: Xiaoming QIAN, Shanghai (CN); Larry Yun FANG, Shanghai (CN); Jiansheng WAN, Shanghai (CN); Zhao LI, Shanghai (CN); Kun LI, Shanghai (CN)

(21) Appl. No.: 17/757,857

(22) PCT Filed: Dec. 24, 2020
(86) PCT No.: PCT/CN2020/138988

§ 371 Date: Jun. 22, 2022

(30) Foreign Application Priority Data
Dec. 25, 2019 (CN) 201911355327.X

(51) Int. Cl. A61K 31/519; A61K 9/20; A61K 9/00; A61K 9/28

(52) U.S. Cl. CPC A61K 31/519; A61K 9/2086; A61K 9/0004; A61K 9/2866

(57) ABSTRACT

Disclosed are a solid preparation, and a preparation method therefor and the use thereof. The solid preparation comprises an immediate-release part and a sustained-release part, which contains active ingredients, wherein the weight ratio of the active ingredient in the sustained-release part to the active ingredient in the immediate-release part is 1:1 or higher. In particular, the solid preparation is a tofacitinib preparation.

The present application claims the priority of the Chinese Application No. 201911355327.X filed on Dec. 25, 2019 with the title of “A solid formulation and a preparation process and use thereof”, of which the contents are incorporated herein by reference in entirety.

### TECHNICAL FIELD

The present disclosure relates to the field of pharmaceutical formulation, and in particular relates to a solid formulation and a preparation process and use thereof.

### BACKGROUND

In the pharmaceutical field, development of a suitable dosage form for active pharmaceutical ingredient (API) is always the focus of the pharmaceutical field. For the dosage form of a drug, the dissolution of a pharmaceutical ingredient can reflect the rate and degree of drug's release in the body, and to a certain extent, the therapeutic effect on the disease.

Taking Tofacitinib as an example, CN103845302A discloses a film-coated tablet of Tofacitinib with good properties. The film-coated tablet contains a film coating base agents, Tofacitinib and a pharmaceutical additive.

For another example, CN104622827A discloses a Tofacitinib tablet and a preparation process thereof. For the tablet, direct compression lactose and microcrystalline cellulose with good fluidity and compression formability are used as diluent agent and dry binder, and direct powder compression method with simple process is adopted for preparation.

CN105878202A discloses a Tofacitinib citrate tablet, which consists of Tofacitinib citrate and a pharmaceutically acceptable excipient, the excipient includes a filler, a disintegrant, a glidant and a coating agent, prepared by direct powder compression method.

CN105101952A discloses an oral sustained release formulation of Tofacitinib and a pharmaceutically acceptable salt thereof, which is a once daily pharmaceutical dosage form, comprising Tofacitinib or a pharmaceutically acceptable salt thereof and a pharmaceutically acceptable carrier, wherein the dosage form is a sustained release dosage form, and the dry granulation compression method is used in Example 1, Example 2 and the like to prepare the tablet core of the sustained release tablet.

### SUMMARY

In one aspect, provided is a solid formulation, wherein the solid formulation comprises an immediate release portion and a sustained release portion, and comprises active ingredients, wherein the weight ratio of the active ingredient in the sustained release portion to the active ingredient in the immediate release portion is about 1:1 or higher.

In an embodiment, in the solid formulation according to the present disclosure, the weight ratio of the active ingredient in the sustained release portion to the active ingredient in the immediate release portion is about 1:1-20:1, preferably about 1:1-9:1, more preferably about 1:1-4:1, specifically preferably about 1:1-7:3, for example about 7:3-4:1.

In a preferable embodiment, the active ingredient is Tofacitinib or a pharmaceutically acceptable salt thereof, particularly Tofacitinib citrate.

In a preferable embodiment, the immediate release portion and the sustained release portion each independently comprise one or more pharmaceutical excipients selected from the group consisting of a sustained release matrix material, a film-forming material, a binder, a filler, a disintegrant, a lubricant.

In a specific embodiment, the solid formulation is in the form of a tablet, a granule, a pellet or a capsule. The tablet is preferably a bilayer tablet, a coated tablet or an osmotic pump tablet.

In another aspect, provided is a pharmaceutical composition, comprising the solid formulation according to the present disclosure. The pharmaceutical composition may comprise one or more pharmaceutically acceptable excipients.

In yet another aspect, provided is use of the solid formulation or the pharmaceutical composition according to the present disclosure for the manufacture of a medicament for treating or preventing a disease. The disease comprises but not limited to lupus, multiple sclerosis, rheumatoid arthritis, psoriatic arthritis, type I diabetes and diabetic complications, cancer, asthma, atopic dermatitis, autoimmune thyroid disease, moderate-to-severe active ulcerative colitis, Crohns' disease, ankylosing spondylitis, juvenile idiopathic arthritis, immune system disorder baldness, vitiligo, systemic lupus erythematosus, Alzheimer's disease and leukemia or the like.

### BRIEF DESCRIPTION OF THE DRAWINGS

FIG. 1 shows the Tofacitinib blood concentration-time curve after the single administration, wherein ● represents the commercial Tofacitinib sustained release tablet, and ▪ represents the Tofacitinib coated tablet according to the present disclosure.

FIG. 2 shows the Tofacitinib blood concentration-time curve after the single administration, wherein ● represents the Tofacitinib coated tablet 1-2 according to the present disclosure, ▪ represents the Tofacitinib coated tablet 1-3 according to the present disclosure, ▴ represents the Tofacitinib coated tablet 1-6 according to the present disclosure.

### DETAILED DESCRIPTION

The present disclosure will be described in detail below, and the description is provided for the purpose of illustration rather than limitation.

### General Definition and Terms

Unless otherwise stated, the technical and scientific terms used herein have the same meaning as commonly understood by a person skilled in the art. If there is a contradiction, the definition provided in this application shall prevail. When expressing a certain amount, concentration or other value or parameter in the form of a range, a preferable range, or a preferable range upper limit and a preferable range lower limit, it should be understood that it corresponds to specifically revealing any range by combining any pair of upper limit of the range or preferable range value with the lower limit of any range or preferable range value, regardless of whether the range is specifically disclosed. Unless otherwise stated, the numerical ranges listed herein are intended to include the endpoints of the range and all integers and fractions (decimals) within the range.

When used with a numerical variable, the term “approximate” or “about” usually refers to the value of the variable and all the values of the variable within the experimental error (for example, within an average 95% confidence interval) or within ±10% of the specified value, or a wider range.

The term “metering ratio” refers to formulating various substances according to a certain weight. For example, the active ingredient and the sustained release material and plasticizer are formulated herein according to the specified weight ratio.

The term “optional” or “optionally” means the event described subsequent thereto may or may not happen. This term encompasses the cases that the event may or may not happen.

The expression “comprise” or its synonyms “contain”, “include”, “have” or the like are meant to be inclusive, which does not exclude other unlisted elements, steps or ingredients. The expression “consist of” excludes any unlisted elements, steps or ingredients. The expression “substantially consist of” refers to specified elements, steps or ingredients within a given range, together with optional elements, steps or components which do not substantively affect the basic and novel feature of the claimed subject matter. It should be understood that the expression “comprise” encompasses the expressions “substantially consist of” and “consist of”.

The term “at least one” or “one or more” as used herein means one, two, three, four, five, six, seven, eight, nine or more.

The term “pharmaceutical acceptable” means application within the scope of sound medical judgment, suitable for use in contact with the tissues of subjects without undue toxicity, irritation, allergic response and the like, having reasonable benefit/risk ratio and effective for intended purpose.

The terms “active pharmaceutical ingredient”, “active ingredient”, “therapeutic agent”, “active substance” or “active agent” refer to a chemical entity which is effective in treating or preventing a target disease or condition. In an embodiment, the active ingredient used in the formulation is Tofacitinib or a pharmaceutically acceptable salt thereof, particularly Tofacitinib citrate. Different portions of the formulations according to the present disclosure may use different forms of the active ingredient. In an embodiment of the present disclosure, the same active ingredient is used. In the present disclosure, a given compound and its corresponding other forms (such as salt, polymorph, solvate, cocrystal, amorphous form, anhydrous form or the like) belong to the same active ingredient, but can be considered as different forms or types.

Those skilled in the art will understand that when different forms or types of an active ingredient exist in a formulation, they need to be converted into their respective free form if the ratio is calculated. Taking Tofacitinib as an example, when Tofacitinib is contained in the immediate release portion and Tofacitinib citrate is contained in the sustained release portion, it is necessary to convert Tofacitinib citrate into Tofacitinib, or to convert Tofacitinib into Tofacitinib citrate. For another example, when the immediate release portion or the sustained release portion contains Tofacitinib and Tofacitinib citrate, it is necessary to convert Tofacitinib citrate into Tofacitinib, or to convert Tofacitinib into Tofacitinib citrate. For another example, when different types of salt forms of an active ingredient coexist in a solid formulation, it is necessary to convert the respective salt forms into corresponding free forms. In addition to the pharmaceutically acceptable salt, an active ingredient such as Tofacitinib, as used herein, also encompasses other forms of the active ingredient, such as polymorph, solvate (including, for example, a hydrate and a mixed solvate and a hydrate of a salt), cocrystal, amorphous form and anhydrous form and a mixture thereof. In a preferable embodiment, the above forms are pharmaceutically acceptable.

The term “dissolution rate” refers to the dissolution rate of a drug from a solid formulation in a specified solvent.

The term “dissolution” refers to the rate and extent of dissolution of a drug from a solid formulation in a specified solvent.

The term “peak time of plasma drug concentration (Tₘₐₓ)” refers to the mean time when peak plasma drug concentration (Cₘₐₓ) is attained after drug administration. The term “peak plasma drug concentration (Cₘₐₓ)” refers to the mean peak plasma drug concentration attained after drug administration. The term “peak time of plasma drug concentration (Tₘₐₓ)” refers to the time when maximum plasma drug concentration (peak plasma drug concentration) is attained after drug administration. The term “AUC₀₋ₜ” refers to the mean integral area under a plasma drug concentration—time curve from the time point of 0 to t after drug administration.

In the present disclosure, the term “Ta value” refers to the time that the blood concentration can be maintained above 17 ng/mL (IC₅₀).

In the present disclosure, the term “strength” refers to the medicine strength, that is, the weight of the active pharmaceutical ingredient contained in each formulation unit, regardless of the form of the active ingredient, and the weight is that of the free form after conversion. Taking Tofacitinib as an example, in a Tofacitinib formulation with strength of 11 mg, when the active ingredient is Tofacitinib, the weight of Tofacitinib is 11 mg. For another example, in a Tofacitinib formulation with strength of 11 mg, when the active ingredient is Tofacitinib citrate, the weight of free Tofacitinib after conversion is 11 mg. For another example, in a Tofacitinib formulation with strength of 11 mg, when the active ingredient is Tofacitinib hydrochloride, the weight of free Tofacitinib after conversion is 11 mg.

### Solid Formulation According to the Present Disclosure

In one aspect, provided is a solid formulation, comprising an immediate release portion and a sustained release portion.

The formulation of the present disclosure also comprises an active ingredient. In the solid formulation of the present disclosure, the active ingredient can each independently be present in the immediate release portion and/or the sustained release portion of the formulation of the present disclosure, respectively, in the same or different forms (e.g., Tofacitinib or a pharmaceutically acceptable salt thereof or the like). For example, the active ingredient can be present in the form of a pharmaceutically acceptable salt in the immediate release portion and the sustained release portion, respectively. For another example, the active ingredient can be present in the immediate release portion in the form of a pharmaceutically acceptable salt and in the sustained release portion in the form of a free base. For another example, citrate salt of tofacitinib as an active ingredient can be present in the immediate release portion and the sustained release portion, respectively. For another example, citrate as an active ingredient can be present in the immediate release portion, and hydrochloride salt of tofacitinib as an active ingredient can be present in the sustained release portion.

As an example of the active ingredient used in the formulation of the present disclosure, Tofacitinib or a pharmaceutically acceptable salt thereof, particularly Tofacitinib citrate, can be used. Therefore, in an embodiment of the present formulation, Tofacitinib or a pharmaceutically acceptable salt thereof (particularly Tofacitinib citrate) can serve as the active pharmaceutical ingredient (API).

Tofacitinib (chemical name of (3R,4R)-4-methyl-3-(methyl-7H-pyrrolo[2,3-d]pyrimidin-4-ylamino)-β-oxo-1-piperidinepropionitrile) is a JAK kinase inhibitor, by inhibiting the JAK pathway, reduces cytokine signaling, cytokine-induced gene expression and cell activation, thereby reducing various chronic inflammatory responses (see the following structure).

As a pharmaceutically acceptable salt of the used active ingredient, it can include, but are not limited to, a salt formed with an inorganic acid, a salt formed with an organic acid, or the like. Non-limiting examples of the salts formed with inorganic acid include but not limited to those formed with hydrochloric acid, hydrobromic acid, nitric acid, sulfuric acid, phosphoric acid or the like. Non-limiting examples of the salts formed with organic acid include but not limited to those formed with formic acid, acetic acid, trifluoroacetic acid, fumaric acid, oxalic acid, malic acid, maleic acid, tartaric acid, citric acid, succinic acid, methanesulfonic acid, benzenesulfonic acid, p-toluenesulfonic acid or the like. In a specifically preferable embodiment, the salt is citrate. In an embodiment of the salt, the active ingredient can be Tofacitinib citrate. Its structure is exemplified as follows:

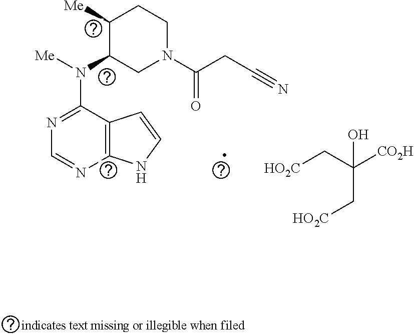 [SMILES: C[C@H]1CCN(C(=O)CC#N)C[C@H]1N(C)c1ncnc2[nH]ccc12 ; O=C(O)CC(O)(CC(=O)O)C(=O)O]

In the formulation of the present disclosure, the active ingredients can be present in the immediate release portion and the sustained release portion. Alternatively, the active ingredients can be present only in the immediate release portion and the sustained release portion. In the latter case, the calculation of the total amount of active ingredients in the formulation can be based on the sum of the active ingredients in the immediate release portion and the sustained release portion (i.e., the sum of the active ingredients in the sustained release portion and the immediate release portion can be 100%).

In the formulation of the present disclosure, the calculation of the active ingredients in the immediate release portion and the sustained release portion can be based on the weight of the active ingredients. Alternatively, as described above, the calculation of the active ingredients should be done according to conversion to a consistent form, and thus such a ratio can also correspond to a molar ratio of the active ingredients.

In an embodiment, the form of the active ingredient in the immediate release portion is identical to that in the sustained release portion, e.g., Tofacitinib citrate.

In an embodiment of the present formulation, the weight ratio of the active ingredient in the sustained release portion to the active ingredient in the immediate release portion is no less than about 1:1. In other words, the amount of the active ingredient in the sustained release portion is greater than or equal to the amount of the active ingredient in the immediate release portion.

In an embodiment, the weight ratio of the active ingredient in the sustained release portion to the active ingredient in the immediate release portion is no greater than about 20:1.

In a preferable embodiment, the weight ratio of the active ingredient in the sustained release portion to the active ingredient in the immediate release portion can be about 1:1-19:1, preferably about 7:3-9:1, about 1:1-4:1, about 7:3-4:1. For example, the weight ratio can be about 1:1, about 1.5:1, about 2:1, about 7:3, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, particularly about 1.5:1, about 4:1, about 7:3, about 9:1, and a range defined by any two of these ratios (values).

In another embodiment, the weight ratio of the active ingredient (e.g., Tofacitinib or a pharmaceutically acceptable salt thereof) in the sustained release portion to the active ingredient (e.g., Tofacitinib or a pharmaceutically acceptable salt thereof) in the immediate release portion can be about 1:1-19:1, for example about 19:1, about 47:3, about 93:7, about 23:2, about 91:9, about 9:1, about 89:11, about 22:3, about 87:13, about 43:7, about 17:3, about 21:4, about 83:17, about 41:9, about 81:19, about 4:1, about 79:21, about 39:11, about 77:23, about 19:6, about 3:1, about 37:13, about 73:27, about 18:7, about 71:29, about 7:3, about 69:31, about 17:8, about 67:33, about 33:17, about 13:7, about 16:9, about 63:37, about 31:19, about 61:39, about 3:2, about 59:41, about 29:21, about 57:43, about 14:11, about 11:9, about 27:23, about 53:47, about 13:12, about 51:49 or about 1:1, and a range defined by any two of these ratios (values).

In a specific embodiment of the formulation of the present disclosure, based on the total weight of the active ingredient in the sustained release portion and the active ingredient in the immediate release portion, the ratio of the active ingredient (e.g., Tofacitinib or a pharmaceutically acceptable salt thereof) in the immediate release portion can be about 5-50%, for example, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49% or about 50%, and a range defined by any two of these ratios (values).

In another embodiment of the present formulation, based on the total weight of the active ingredient in the sustained release portion and the active ingredient in the immediate release portion, the ratio of the active ingredient (e.g., Tofacitinib or a pharmaceutically acceptable salt thereof) in the sustained release portion can be about 50-95%, for example about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94% or about 95%, and a range defined by any two of these ratios (values).

In an embodiment, the weight ratio of the active ingredient (e.g., Tofacitinib or a pharmaceutically acceptable salt thereof) in the sustained release portion to the active ingredient (e.g., Tofacitinib or a pharmaceutically acceptable salt thereof) in the immediate release portion is about 7:3-9:1, for example about 9:1, about 89:11, about 22:3, about 87:13, about 43:7, about 17:3, about 21:4, about 83:17, about 41:9, about 81:19, about 4:1, about 79:21, about 39:11, about 77:23, about 19:6, about 3:1, about 37:13, about 73:27, about 18:7, about 71:29 or about 7:3, and a range defined by any two of these ratios (values).

In a specific embodiment, based on the total weight of the active ingredient in the sustained release portion and the active ingredient in the immediate release portion, the ratio of the active ingredient (e.g., Tofacitinib or a pharmaceutically acceptable salt thereof) in the immediate release portion is about 10-30%, for example about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29% or about 30%, and a range defined by any two of these ratios (values).

In another embodiment, based on the total weight of the active ingredient in the sustained release portion and the active ingredient in the immediate release portion, the ratio of the active ingredient (e.g., Tofacitinib or a pharmaceutically acceptable salt thereof) in the sustained release portion is about 70-90%, for example about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89% or about 90%, and a range defined by any two of these ratios (values).

In an embodiment, the weight ratio of the active ingredient (e.g., Tofacitinib or a pharmaceutically acceptable salt thereof) in the sustained release portion to the active ingredient (e.g., Tofacitinib or a pharmaceutically acceptable salt thereof) in the immediate release portion is about 39:11-41:9, for example about 41:9, about 81:19, about 4:1, about 79:21, about 39:11, and a range defined by any two of these ratios (values).

In a specific embodiment, based on the total weight of the active ingredient in the sustained release portion and the active ingredient in the immediate release portion, the ratio of the active ingredient (e.g., Tofacitinib or a pharmaceutically acceptable salt thereof) in the immediate release portion is about 18-22%, for example about 18%, about 19%, about 20%, about 21% or about 22%.

In another embodiment, based on the total weight of the active ingredient in the sustained release portion and the active ingredient in the immediate release portion, the ratio of the active ingredient (e.g., Tofacitinib or a pharmaceutically acceptable salt thereof) in the sustained release portion is about 78-82%, for example about 78%, about 79%, about 80%, about 81% or about 82%.

In a specific embodiment, the weight ratio of the active ingredient (e.g., Tofacitinib or a pharmaceutically acceptable salt thereof) in the sustained release portion to the active ingredient (e.g., Tofacitinib or a pharmaceutically acceptable salt thereof) in the immediate release portion is about 39:11-41:9, for example about 4:1. In a specific embodiment, based on the total weight of the active ingredient in the sustained release portion and the active ingredient in the immediate release portion, the ratio of the active ingredient (e.g., Tofacitinib or a pharmaceutically acceptable salt thereof) in the sustained release portion is about 78-82%, for example about 80%.

### Form and Composition of the Formulation

The formulation of the present disclosure can be in the form of a granule, a pellet, a tablet or a capsule or the like, or a combination of the above formulations.

In an embodiment of the formulation of the present disclosure, the sustained release portion and the immediate release portion can each independently be in the form of a granule, a pellet, a layer, a tablet (such as an osmotic pump tablet), a coating. As an example, the immediate release portion can also be in the form of a granule or a particulate comprised in the sustained release portion such as a sustained coating.

The formulation of the present disclosure can contain one or more sustained release portions and one or more immediate release portions. These sustained release portions and immediate release portions as well as the active ingredients and other ingredients therein can be independently selected.

Those skilled in the art will understand that the sustained release portion and immediate release portion as used herein should be understood broadly and not referring to only one specific component in the formulation. For example, a formulation such as a coated tablet or an osmotic pump tablet can contain a sustained tablet core and a coating thereon. When the coating is a sustained coating, it can also be considered a sustained release portion.

In an embodiment, the sustained release portion in the formulation is a sustained release granule. In another embodiment, the immediate release portion in the formulation is an immediate release granule.

In a preferrable embodiment, the immediate release granule and the sustained release granule can be filled in a capsule. In another preferrable embodiment, the immediate release granule and the sustained release granule can be filled in a pouch.

In an embodiment, the immediate release granule and the sustained release granule can be mixed and compressed into a tablet.

In an embodiment, the sustained release portion in the formulation is a coating sustained release granule or a matrix sustained release granule. In another embodiment, the immediate release portion in the formulation is located within or outside a coating film or outside a matrix sustained release granule.

In an embodiment, the sustained release portion in the formulation is a sustained release pellet. In another embodiment, the immediate release portion in the formulation is an immediate release pellet.

In a preferable embodiment, the immediate release pellet and the sustained release pellet can be filled in a capsule.

In a preferred embodiment, the immediate release pellet and the sustained release pellet can be filled in a pouch. In an embodiment, the immediate release pellet and the sustained release pellet can be mixed and compressed into a tablet.

In a specific embodiment, the formulation of the present disclosure may comprise a sustained release pellet, wherein the sustained release pellet is a coated sustained release pellet or a matrix sustained release pellet, and the immediate release portion is located inside or outside the coating film or outside the matrix sustained release pellet.

In an embodiment, the formulation of the present disclosure can be a bilayer tablet, wherein one layer is a sustained release layer (corresponding to the sustained release portion), and the other layer is an immediate release layer (corresponding to the immediate release portion). In a preferable embodiment, the sustained release layer is a matrix sustained release tablet. In another preferable embodiment, the sustained release layer is a membrane-controlled sustained release tablet. In a specifically preferable embodiment, the sustained release layer is an osmotic pump tablet.

In an embodiment, the formulation of the present disclosure can be a coated tablet, wherein the sustained release portion is a coated tablet core or a matrix tablet core, and the immediate release portion is located within or outside the coating film or outside the matrix tablet core. In a preferable embodiment, the coated tablet is an osmotic pump tablet.

In an embodiment, the formulation of the present disclosure can be a capsule, the content of which is a sustained release granule or pellet, and the immediate release portion is coated around the capsule or located within the capsule shell.

In a preferrable embodiment of the present formulation, the formulation is a coated tablet, wherein the sustained release portion is a coated tablet core, and the immediate release portion is a coating film, wherein the coated tablet core and coating film each independently contain the active ingredient of the present disclosure. Preferably, the coated tablet is an osmotic pump tablet.

In a preferable embodiment of the present formulation, the active ingredient can be Tofacitinib or a pharmaceutically acceptable salt thereof, particularly Tofacitinib citrate.

In an embodiment of the present formulation, the sustained release portion comprises a sustained release matrix material, which can be one or more selected from the group consisting of: hydroxypropyl methyl cellulose, hydroxypropyl cellulose, hydroxyethyl cellulose, polyoxyethylene, glyceryl behenate, polymethylmethacrylate, acrylic resin, povidone, xanthan gum, sodium alginate, calcium alginate, carbomer, chitin, polyethylene, polysiloxane.

The followings are examples of some available sustained release matrix materials, without limitation:

hydroxypropyl methyl cellulose, which can be selected from for example the commercial product Metolose SR 90SH of Shin-Etsu;

hydroxypropyl cellulose, which can be selected from for example the commercial products Klucel™ GF, Klucel™ JF, Klucel™ MF, Klucel™ HF of Ashland;

hydroxyethyl cellulose, which can be selected from for example commercial products Natrosol G, Natrosol M, Natrosol HX, Natrosol HHX of Ashland;

glyceryl behenate, which can be selected from for example the commercial product Compritol® AT0888 of GATTEFOSSE.

In an embodiment, the sustained release portion comprises a sustained release matrix material. Preferably, the sustained release matrix material is about 40%-80%, more preferably about 40%-50% or about 50%-70%, for example about 40%, about 45%, about 50%, about 55%, about 60%, about 65%, about 70%, about 75% or about 80% of the weight of the sustained release portion.

In an embodiment, the sustained release portion and/or the immediate release portion comprises a film-forming material. The film-forming material can be one or more selected from the group consisting of: cellulose acetate, ethyl cellulose, hydroxypropyl methyl cellulose, acrylic resins.

The following are examples of some available film-forming materials, without limitation: ethyl cellulose, which can be selected from for example the commercial products Aqualon™ ethyl cellulose of Ashland, ETHOCEL™ of Dupont;

hydroxypropyl methyl cellulose, which can be selected from for example the commercial product Metolose 60 SH of Shin-Etsu;

cellulose acetate, which can be selected from for example the commercial products CA-320S, CA-394-60LF, CA-398-3, CA-398-6, CA-398-10 and CA-398-30 of Eastman;

ethyl cellulose, which can be selected from for example the ETHOCEL™ Standard 10 Premium of Dupont.

In an embodiment, the sustained release portion comprises a film-forming material (membrane). The active ingredient may or may not be located in the film-forming material. The active ingredient may be an immediate release active ingredient or may be a sustained release active ingredient.

When the sustained release portion comprises a film-forming material, the film-forming material can be about 30%-70%, more preferably about 40%-67%, for example about 30%, about 35%, about 40%, about 45%, about 50%, about 55%, about 60%, about 67% of the weight of the sustained release portion.

It should be understood that when a formulation comprises a sustained release portion or an immediate release portion which are morphologically separated, the content of the material should be calculated based on the portion where the material is located. For example, the coated tablet or osmotic pump tablet can comprise a sustained release tablet core and/or a sustained coating. When the tablet core or coating comprises a material such as a film-forming material or a matrix material, the content should be calculated based on the corresponding tablet core or coating.

In an embodiment, the immediate release portion comprises a film-forming material (membrane). Preferably, the film-forming material is about 40%-80%, more preferably about 50%-70%, for example about 40%, about 45%, about 50%, about 55%, about 60%, about 65%, about 70%, about 75% or about 80% of the weight of the immediate release portion.

In an embodiment, the formulation of the present disclosure, in the sustained release portion, the immediate release portion, or another portion beyond them, can also optionally comprise one or more of the following pharmaceutically acceptable excipients: a binder, a filler, a disintegrant, a lubricant or the like. The amount of these pharmaceutically acceptable excipients can be selected by those skilled in the art according to needs.

The following are examples of some pharmaceutically acceptable excipients, without limitation:

The binder can be one or more selected from the group consisting of polyethylene glycol, starch, hydroxypropyl cellulose, hydroxypropyl methyl cellulose, pregelatinized starch, polyvinylpyrrolidone, gum arabic and gelatin, preferably starch, pregelatinized starch, modified starch, microcrystalline cellulose, silicified microcrystalline cellulose, low-substituted hydroxypropyl cellulose glucose, sucrose, lactose, sorbitol, mannitol, erythritol, calcium carbonate and calcium hydrophosphate, particularly preferably lactose, mannitol, microcrystalline cellulose and silicified microcrystalline cellulose.

The disintegrant can be one or more selected from the group consisting of microcrystalline cellulose, carboxymethyl cellulose, copovidone, croscarmellose sodium, calcium carboxymethylcellulose, sodium carboxymethyl starch, low-substituted hydroxypropyl cellulose, hydroxymethyl starch, alginic acid, sodium alginate, guar gum, corn starch and magnesium aluminum silicate, preferably copovidone and croscarmellose sodium.

The lubricant can be one or more selected from the group consisting of magnesium stearate, stearic acid, stearate, sodium stearyl fumarate, sodium lauryl sulfate, polyethylene glycol, sodium benzoate, sucrose fatty acid ester, micronized silica gel, talcum powder, glyceryl monostearate, glyceryl behenate, glyceryl palmitostearate, stearic acid and hydrogenated vegetable oil, preferably magnesium stearate.

It should be understood that the above-listed pharmaceutically acceptable excipients are only illustrative and exemplary. Therefore, the pharmaceutical formulation of the present disclosure is not limited to comprise only the pharmaceutically acceptable excipients as listed above. Those skilled in the art can use conventional techniques to change or modify the above-mentioned excipients with equivalence without departing the protection scope of the present disclosure.

In another aspect, provided is a pharmaceutical composition comprising the solid formulation of the present disclosure. The pharmaceutical composition can also comprise one or more pharmaceutically acceptable auxiliaries.

### Release of the Formulation of the Disclosure

By including an immediate release portion and a sustained release portion, the formulation of the present disclosure can exhibit a combination of immediate release and sustained release dual release mode, enabling the rapid onset of the pharmaceutical ingredient while providing an effective and stable blood concentration for a longer period of time.

In an embodiment, based on the total weight of the active ingredient in the solid formulation, the active ingredient is released in an amount of about 5%-50% within 15 min, for example about 5%, about 10%, about 15%, about 20%, about 25%, about 30%, about 45% or about 50%. In a preferable embodiment, based on the total weight of the active ingredient in the solid formulation, the active ingredient is released in an amount of about 10%-30% within 15 min, for example about 10%, about 15%, about 20%, about 25% or about 30%, particularly about 20%.

In an embodiment of the present formulation, the active ingredient in the immediate release portion has a dissolution rate within 15 min of about 0.03-0.37 mg/min, for example, about 0.07 mg/min, about 0.13 mg/min, about 0.15 mg/min, about 0.17 mg/min, about 0.21 mg/min, about 0.24 mg/min, about 0.28 mg/min or about 0.33 mg/min. In a preferable embodiment of the present formulation, the active ingredient in the immediate release portion has a dissolution rate within 15 min of about 0.07-0.22 mg/min, for example, about 0.07 mg/min, about 0.13 mg/min, about 0.15 mg/min, about 0.17 mg/min or about 0.21 mg/min, specifically preferably about 0.15 mg/min.

In another embodiment of the present formulation, the ratio of the average dissolution rate of the active ingredient in the immediate release portion within 15 min to the average dissolution rate of the active ingredient in the sustained release portion over the entire release time range is about 0.5:1-100:1, for example about 1.3:1, about 10.4:1, about 30:1, about 64.5:1 or about 96.5:1. In a preferable embodiment, the ratio is about 1.4:1-11:1, for example about 1.6:1, about 3.3:1, about 6.7:1, about 8.5:1 or 10:1. In a specifically preferable embodiment, the ratio is about 3:1-6:1, for example about 3.3:1, about 3.9:1, about 4.5:1, about 5.2:1 or about 5.8:1.

The dissolution rate and release amount can be calculated from the dissolution-time curve of the active ingredient. The dissolution can be determined by conventional methods in the field, for example, according to the USP II method (Paddle Method) of United States Pharmacopeia.

In an embodiment, the active ingredient in the formulation according to the present disclosure has a total release amount within 1 h of about 20%-65%, for example about 20%, about 25%, about 30%, about 35%, about 40%, about 45%, about 50%, about 55%, about 60% or about 65%. In a preferable embodiment, the release amount is about 30%-50%, for example about 30%, about 35%, about 40%, about 45% or about 50%.

In an embodiment, the active ingredient in the formulation according to the present disclosure has a total release amount within 3 h of about 55%-95%, for example about 55%, about 60%, about 65%, about 70%, about 75%, about 80%, about 85%, about 90% or about 95%. In a preferable embodiment, the release amount is about 60%-80%, for example about 60%, about 65%, about 70%, about 75% or about 80%.

In an embodiment, the active ingredient in the formulation according to the present disclosure has a total release amount within 5 h of no less than about 80%, for example about 80%, about 85%, about 90%, about 95% or about 99% or higher. In a preferable embodiment, the release amount is no less than about 85%, for example about 85%, about 90%, about 95%, about 96% or about 97% or higher.

In an embodiment, the active ingredient in the formulation according to the present disclosure has a total release amount within 15 min of about 5%-50%, the total release amount within 1 h is about 20%-65%; the total release amount within 3 h is about 55%-90%; and the total release amount within 5 h is no less than about 80%.

The calculations of above-mentioned release amount are based on the total amount of active ingredient in the formulation prior to release.

In an embodiment, after a single administration, the time required by the formulation according to the present disclosure for achieving a blood concentration of 17 ng/ml is about 10-60 min, for example about 15 min, about 20 min, about 25 min, about 30 min, about 35 min, about 40 min, about 45 min, about 50 min, about 55 min or about 60 min. In a preferable embodiment, after a single administration, the time required by the formulation according to the present disclosure for achieving a blood concentration of 17 ng/ml is about 25-45 min, for example about 25 min, about 30 min, about 35 min, about 40 min, about 45 min or about 50 min. In a specifically preferable embodiment, after a single administration, the time required by the formulation according to the present disclosure for achieving a blood concentration of 17 ng/ml is about 20-30 min, for example about 20 min, about 22 min, about 24 min, about 26 min, about 28 min or about 30 min.

In an embodiment, after a single administration, the duration time of the solid formulation according to the present disclosure for achieving a blood concentration over 17 ng/ml is about 8-12 h (for example about 8, about 8.5, about 9, about 9.5, about 10, about 10.5, about 11, about 11.5 or about 12 h), preferably about 9-11 h.

The blood concentration of 17 ng/ml herein corresponds to the half inhibitory concentration of JAK1/3 in vitro (i.e., IC₅₀ value).

As used herein, “single administration” refers to a solid formulation of one dosage unit (for example, one tablet or one pill) containing 11 mg or 22 mg of the active ingredient (e.g., Tofacitinib citrate) for one administration. The administration frequency can be, for example, once daily. The administration subject can be a mammal, for example a human.

In a preferrable embodiment of the formulation herein, the active ingredient can be Tofacitinib or a pharmaceutically acceptable salt thereof, particularly Tofacitinib citrate.

### Preparation Process According to the Present Disclosure

Provided is also a process for preparing the solid formulation according to the present disclosure, comprising

1) combining the active ingredient with one or more pharmaceutically acceptable excipients to give the immediate release portion,

2) combining the active ingredient with one or more pharmaceutically acceptable excipients to give the sustained release portion,

3) combing the immediate release portion and the sustained release portion to give the solid formulation;

wherein

step 1) and step 2) are conducted in any order independently;

the active ingredients and the excipients in step 1) and step 2) are used independently.

In an embodiment, the active ingredients in step 1) and step 2) are each independently Tofacitinib or a pharmaceutically acceptable salt thereof, particularly Tofacitinib citrate.

In another embodiment, the total amount of the active ingredients in step 1) and step 2) is 100%. In other words, the active ingredient in the formulation is only present in the immediate release portion and/or sustained release portion.

The immediate release portion, sustained release portion, active ingredient, excipient in the formulation of the present disclosure can be referred to the corresponding descriptions above.

In an exemplary embodiment, a pressing method can be used, comprising:

1. Preparing the immediate release layer granules: Mixing the active ingredient, the filler, the disintegrant and half of the lubricant according to the metering ratio uniformly; using a dry granulator to prepare the above mixture into granules, and then mixing them with the remaining half of the lubricant to give the blended granules of the immediate release layer.

2. Preparing the sustained release layer granules: Mixing the active ingredient, the filler, the sustained release matrix material and half of the lubricant according to the metering ratio uniformly; using a dry granulator to prepare the above mixture into granules, and then mixing them with the remaining half of the lubricant to give the blended granule of the sustained release layer.

3. Compression: Pressing the immediate release layer blended granules and the sustained release layer blended granules into a bilayer tablet by using a bilayer tableting machine to give the final bilayer tablet.

4. Coating: If desired, adding a protective coating commonly used in the art. The coating may not contain the active ingredient.

In an embodiment, the process for preparing a coated tablet is to press a sustained release tablet, and then apply an immediate release coating containing the active ingredient. Such a process for preparing the coated tablet comprises the following steps:

1. Preparing sustained release granules: Mixing the active ingredient, the filler, the sustained release matrix material and half of the lubricant according to the metering ratio uniformly; using a dry granulator to prepare the above mixture into granules and mix them with the remaining half of the lubricant to give the blended granules.

2. Compression: Pressing the above blended granules into a sustained release tablet using a tableting machine.

3. Coating: Mixing the active ingredient, the filler and the membrane uniformly, which are added into an isopropanol solution containing 10 wt % purified water with sufficiently and uniformly stirring and prepared to be a suspension with a solid content of about 7%. Spraying the above suspension coating on the sustained release tablet, which after drying is prepared into a coated tablet.

In an embodiment, the process for preparing a composition containing the active ingredient is to prepare immediate release pellets by extrusion spheronization, coating a part of the pellets into sustained release pellets, and filling both of them into a capsule in proportion. Such a process for preparing a capsule comprises the following steps:

1. Preparing the immediate release pellets: Mixing the active ingredient, the filler, the binder and the lubricant according to the metering ratio uniformly, to which is added an appropriate amount of purified water to prepare a soft material, extruding the above soft material using an extruding spheronizer into wet drug-containing immediate release pellets, and then drying the above pellets using a fluidized bed to give immediate release pellets.

2. Preparing sustained release pellets: Adding the sustained coating membrane and the filler into an isopropanol solution containing 10 wt % purified water with sufficiently and uniformly stirring which is prepared into a suspension with a solid content of about 7%. Placing a part of the immediate release pellets according to the metering ratio into a fluidized bed, coating the above solution on the surface of the immediate release pellets, and preparing the sustained release pellet after drying.

3. Capsules filling: filling the immediate release pellets and the sustained release pellets into capsules using a capsule filling machine according to the respective weights to give capsules.

It should be understood that the preparation process of the composition listed above is only illustrative and exemplary. Therefore, the preparation process of the composition of the present disclosure is not limited that as listed above. Those skilled in the art can use conventional techniques to change or modify the preparation process with equivalence without departing the scope of the present disclosure.

### Pharmaceutical Method and Pharmaceutical Use

Provided is also a method for preventing and treating a disease, comprising administering to a subject in need thereof an effective amount of the solid formulation or pharmaceutical composition of the present disclosure.

The solid formulation or pharmaceutical composition of the present disclosure can be useful for preventing and treating a disease.

Provided is also use of the solid formulation or the pharmaceutical composition according to the present disclosure for the manufacture of a medicament for treating or preventing a disease.

In an embodiment, the disease comprises lupus, multiple sclerosis, rheumatoid arthritis, psoriatic arthritis, type I diabetes and diabetic complications, cancer, asthma, atopic dermatitis, autoimmune thyroid disease, moderate-to-severe active ulcerative colitis, Crohns' disease, ankylosing spondylitis, juvenile idiopathic arthritis, immune system disorder baldness, vitiligo, systemic lupus erythematosus, Alzheimer's disease, leukemia or the like.

### Beneficial Effect

Compared with the prior art, the solid formulation of the present disclosure comprises an immediate release portion and a sustained release portion. On the basis that the immediate release achieves the potency, it also has a sustained release effect, such that the potency can rapidly onset and be maintained for a long time.

In particular, in the coated tablet and osmotic pump tablet of the present disclosure, the tablet core is the sustained release portion, and the outer coating also contains a certain amount of active ingredient. As the immediate release portion of the dual-release formulation, the release rate of the active ingredient in the early stage is accelerated, eliminating the delayed release phenomenon in the prior art. Meanwhile, the sustained release portion can achieve stable blood concentration for a long time.

Further, the immediate release portion of the formulation of the present disclosure has a faster release rate, where within 15 min, the dissolution rate of the active ingredient (e.g., Tofacitinib or a pharmaceutically acceptable salt thereof) can reach about 0.03 mg/min-0.37 mg/min. The dissolution rate of the active ingredient can be changed by adjusting the contents of the active ingredient (e.g., Tofacitinib or a pharmaceutically acceptable salt thereof) in the immediate release portion and the active ingredient (e.g., Tofacitinib or a pharmaceutically acceptable salt thereof) in the sustained release portion to meet the requirements of different medication environments and patients with different conditions.

In another aspect, in the formulation of the present disclosure, the dissolution rate ratio of the active ingredient (e.g., Tofacitinib or a pharmaceutically acceptable salt thereof) in the immediate release portion and the active ingredient (e.g., Tofacitinib or a pharmaceutically acceptable salt thereof) in the sustained release portion within 15 min is about 0.5:1-100:1. The dissolution rate ratio can be changed by adjusting the contents of the active ingredient in immediate release portion and the active ingredient in the sustained release portion to achieve immediate release or sustained release of the active ingredient to meet the requirements of different medication environments and patients with different conditions.

In addition, the formulation of the present disclosure can provide longer duration of single administration blood concentration above 17 ng/ml, for example about 9-11 h. Compared to commercial formulations, the duration is longer, thereby allowing the drug to remain stable concentration fluctuations in the body so as to achieve dosing once daily and provide fast and durable disease management.

### Examples

The technical solutions of the present disclosure will be further described below by reference to specific examples. It should be noted that the examples are exemplary only, rather than any limitation to the protection scope of present disclosure. The present disclosure can also have other embodiments or be practiced with various means. Unless otherwise stated, all percentages, parts, ratios or the like herein are provided on a weight basis. Unless otherwise stated, the apparatus, reagents, raw materials or the like herein are commercially available.

The sources of some of the materials used are listed below: Tofacitinib citrate, purchased from Haiyou Freda; glyceryl behenate, purchased from GATTEFOSSE, Compritol AT0888; hydroxyethyl cellulose, purchased from Ashland, Natrosol HX; hydroxypropyl methyl cellulose, purchased from Shin-Etsu, Metolose SR 90SH; cellulose acetate, purchased from Eastman, CA-398-10.

In the following examples, the formulations were tested for dissolution by the USP II method (Paddle Method) of United States Pharmacopeia. The specific conditions were as follows: phosphate buffer with pH 6.8 was used as the dissolution medium, the volume of the dissolution medium was 900 ml, the rotational speed of the dissolution assay was 50 rpm, the assay temperature was 37° C.

The parameters of the dissolution assay method used are as follows.

| Dissolution method | USP II method (Paddle Method) | |
|---|---|---|
| Dissolution medium | phosphate buffer, pH 6.8 | |
| medium volume | 900 | ml |
| Rotary speed | 50 | rpm |

| | |
|---|---|
| Temperature | 37° C. |

Dissolution sample analysis: The solution obtained in the dissolution assay was filtered with a 0.45 μm filtration membrane to collect the filtrate, which was measured by high performance liquid chromatography at a wavelength of 210 nm with Agilent 1200.

### Example 1. Preparation of Bilayer Tablets

1. Preparation:

Preparation was conducted according to the specific compositions and amounts (parts by weight, hereafter) in Table 1.

Immediate release layer granules: According to the metering ratio, Tofacitinib, the filler, the disintegrant and half amount of lubricant were mixed uniformly using a hopper mixer (Nantong BEITE HBD-200); the above mixture was prepared into granules using a dry granulator (Alexanderwerk WP120, Germany), which were mixed with the remaining half amount of lubricant using a hopper mixer (Nantong BEITE HBD-200) to give the blended granules of the immediate release layer.

Sustained release layer granule: According to the metering ratio, Tofacitinib, the filler, the sustained release matrix material and half amount of lubricant were mixed uniformly using a hopper mixer (Nantong BEITE HBD-200); the above mixture was prepared into granules using a dry granulator (Alexanderwerk WP120, Germany), which were mixed with the remaining half amount of the lubricant using a hopper mixer (Nantong BEITE HBD-200) to give the blended granules of the sustained release layer.

Compression: The immediate release layer blended granules and the sustained release layer blended granules were pressed into bilayer tablets using a bilayer tableting machine (FETTE P3030) to give the final bilayer tablets.

According to the above procedure, the bilayer tablets 1-1 to 1-10 of the present disclosure were prepared.

TABLE 1
Specific composition of bilayer tablets (parts by weight)

| | | bilayer | bilayer | bilayer | bilayer | bilayer | bilayer | bilayer | bilayer | bilayer | bilayer |
|---|---|---|---|---|---|---|---|---|---|---|---|
| | | tablet | tablet | tablet | tablet | tablet | tablet | tablet | tablet | tablet | tablet |
| Component | Function | 1-1 | 1-2 | 1-3 | 1-4 | 1-5 | 1-6 | 1-7 | 1-8 | 1-9 | 1-10 |

Immediate release layer

| | | | | | | | | | | | |
|---|---|---|---|---|---|---|---|---|---|---|---|
| Tofacitinib | active | 0.5 | 1 | 2 | 3 | 4 | 5 | 2 | 2 | 2 | 2 |
| citrate | ingredien | | | | | | | | | | |
| microcrystalline | filler | 138.2 | 138 | 137.5 | 137 | 136.5 | 136 | 143.5 | 127.5 | 117.5 | 107.5 |
| cellulose | | | | | | | | | | | |
| lactose | filler | 138.3 | 138 | 137.5 | 137 | 136.5 | 136 | 143.5 | 127.5 | 117.5 | 107.5 |
| copovidone | disintegrant | 20 | 20 | 20 | 20 | 20 | 20 | 8 | 40 | 60 | 80 |
| magnesium | lubricant | 3 | 3 | 3 | 3 | 3 | 3 | 3 | 3 | 3 | 3 |
| stearate | | | | | | | | | | | |

Sustained release layer

| | | | | | | | | | | | |
|---|---|---|---|---|---|---|---|---|---|---|---|
| Tofacitinib | active | 9.5 | 9 | 8 | 7 | 6 | 5 | 8 | 8 | 8 | 8 |
| citrate | ingredien | | | | | | | | | | |
| microcrystalline | filler | 93.2 | 93.5 | 94 | 94.5 | 95 | 95.5 | 114 | 94 | 74 | 34 |
| cellulose | | | | | | | | | | | |
| lactose | filler | 93.3 | 93.5 | 94 | 94.5 | 95 | 95.5 | 114 | 94 | 74 | 34 |
| glyceryl | sustained | 200 | 200 | 200 | 200 | 200 | 200 | 160 | 200 | 240 | 320 |
| behenate | release | | | | | | | | | | |
| | matrix | | | | | | | | | | |
| | material | | | | | | | | | | |
| magnesium | lubricant | 4 | 4 | 4 | 4 | 4 | 4 | 4 | 4 | 4 | 4 |
| stearate | | | | | | | | | | | |

2. Dissolution Assay of the Formulations

According to the procedure described above, the dissolutions of each bilayer tablets in phosphate buffer, pH 6.8 were determined, and the results were shown in Table 2.

TABLE 2
Dissolutions of bilayer tablets

| | Dissolution (%) | | | | | | | |
|---|---|---|---|---|---|---|---|---|
| Time point (h) | 0.25 | 1 | 2 | 3 | 4 | 5 | 6 | 7 |
| bilayer tablet 1-1 | 5 | 27 | 45 | 58 | 69 | 80 | 89 | 96 |
| bilayer tablet 1-2 | 10 | 31 | 48 | 60 | 71 | 81 | 90 | 96 |
| bilayer tablet 1-3 | 20 | 39 | 54 | 65 | 74 | 83 | 91 | 97 |
| bilayer tablet 1-4 | 30 | 46 | 59 | 69 | 77 | 85 | 92 | 99 |
| bilayer tablet 1-5 | 40 | 54 | 65 | 73 | 80 | 87 | 93 | 99 |
| bilayer tablet 1-6 | 50 | 62 | 71 | 78 | 84 | 89 | 94 | 100 |
| bilayer tablet 1-7 | 20 | 42 | 64 | 82 | 99 | 99 | 100 | 100 |
| bilayer tablet 1-8 | 20 | 37 | 50 | 61 | 72 | 83 | 92 | 99 |
| bilayer tablet 1-9 | 20 | 33 | 45 | 54 | 63 | 72 | 81 | 90 |
| bilayer tablet 1-10 | 20 | 27 | 37 | 46 | 52 | 59 | 65 | 68 |

From the data in Table 2, it can be seen that the bilayer tablets prepared with different compositions had wide drug release ranges. The release amount in 15 min was the content of the active ingredient Tofacitinib in the immediate release layer. The results showed that the immediate release layers with different compositions released the drugs within 15 min completely. In the subsequent process, the solid formulations can also continuously release the active ingredient Tofacitinib at a steady rate, providing effective and stable blood concentrations for a longer time. In particular, the release of formulations 1-1 to 1-8 was completed within 7 h, showing a more advantageous release effect.

### Example 2 Preparation of Coated Tablets

1. Preparation:

Preparation was conducted according to the specific compositions and amounts (parts by weight, hereafter) in Table 3.

Sustained release granule: According to the metering ratio, Tofacitinib, the filler, the sustained release matrix material and half amount of lubricant were mixed uniformly using a hopper mixer (Nantong BEITE HBD-200); the above mixture was prepared into granules using a dry granulator (Alexanderwerk WP120, Germany), which were mixed with the remaining half amount of lubricant using a hopper mixer (Nantong BEITE HBD-200) to give the blended granules.

Compression: The above blended granules were pressed into sustained release tablets using a tableting machine (FETTE P2020).

Coating: Tofacitinib and membrane material were mixed uniformly, then added to an isopropanol solution containing 10 wt % purified water with sufficiently and uniformly stirring and prepared to be a suspension with a solid content of about 7%. The above suspension was sprayed onto sustained release tablets using a coating pan (O'Hara Lab coat M), which were dried to give coated tablets. During the spraying process, the material temperature was 25-35° C., and the inlet air volume was maintained as 50-100 cm³/h. After the spraying was completed, the coated tablets of Tofacitinib were dried in a 30° C. coating machine for 2 h, until the loss on drying was less than 2.0%, to give the final coated tablets.

According to the above procedure, the coated tablets 1-1 to 1-10 of the present disclosure were prepared.

TABLE 3
Specific composition of coated tablets (parts by weight)

| | | coated | coated | coated | coated | coated | coated | coated | coated | coated | coated |
|---|---|---|---|---|---|---|---|---|---|---|---|
| | | tablet | tablet | tablet | tablet | tablet | tablet | tablet | tablet | tablet | tablet |
| Component | Function | 1-1 | 1-2 | 1-3 | 1-4 | 1-5 | 1-6 | 1-7 | 1-8 | 1-9 | 1-10 |

Sustained release tablet core

| | | | | | | | | | | | |
|---|---|---|---|---|---|---|---|---|---|---|---|
| Tofacitinib | active | 9.5 | 9 | 8 | 7 | 6 | 5 | 8 | 8 | 8 | 8 |
| citrate | ingredient | | | | | | | | | | |
| microcrystalline | filler | 93.2 | 93.5 | 94 | 94.5 | 95 | 95.5 | 114 | 94 | 74 | 34 |
| cellulose | | | | | | | | | | | |
| lactose | filler | 93.3 | 93.5 | 94 | 94.5 | 95 | 95.5 | 114 | 94 | 74 | 34 |
| glyceryl | sustained | 200 | 200 | 200 | 200 | 200 | 200 | 160 | 200 | 240 | 320 |
| behenate | release | | | | | | | | | | |
| | matrix | | | | | | | | | | |
| | material | | | | | | | | | | |
| magnesium | lubricant | 4 | 4 | 4 | 4 | 4 | 4 | 4 | 4 | 4 | 4 |
| stearate | | | | | | | | | | | |

Coating prescription

| | | | | | | | | | | | |
|---|---|---|---|---|---|---|---|---|---|---|---|
| Tofacitinib | active | 0.5 | 1 | 2 | 3 | 4 | 5 | 2 | 2 | 2 | 2 |
| citrate | ingredient | | | | | | | | | | |
| ethyl | membrane | 10 | 10 | 10 | 10 | 10 | 10 | 4 | 20 | 30 | 40 |
| cellulose | material | | | | | | | | | | |
| mannitol | filler | 10 | 10 | 10 | 10 | 10 | 10 | 4 | 20 | 30 | 40 |
| 90% | solvent | appro- | appro- | appro- | appro- | appro- | appro- | appro- | appro- | appro- | appro- |
| isopropanol* | | priate | priate | priate | priate | priate | priate | priate | priate | priate | priate |
| | | amount | amount | amount | amount | amount | amount | amount | amount | amount | amount |

*volatilized during preparation.

2. Dissolution Assay of the Formulations

According to the procedure described above, the dissolutions of each coated tablets in phosphate buffer, pH 6.8 were determined, and the results were shown in Table 4.

TABLE 4
Dissolutions of coated tablets

| | Dissolution (%) | | | | | | | |
|---|---|---|---|---|---|---|---|---|
| Time point (h) | 0.25 | 1 | 2 | 3 | 4 | 5 | 6 | 7 |
| coated tablet 1-1 | 5 | 20 | 40 | 55 | 69 | 80 | 89 | 96 |
| coated tablet 1-2 | 10 | 24 | 43 | 57 | 71 | 81 | 90 | 96 |
| coated tablet 1-3 | 20 | 33 | 49 | 62 | 74 | 83 | 91 | 97 |
| coated tablet 1-4 | 30 | 41 | 56 | 67 | 77 | 85 | 92 | 97 |
| coated tablet 1-5 | 40 | 49 | 62 | 72 | 80 | 87 | 93 | 97 |
| coated tablet 1-6 | 50 | 58 | 68 | 76 | 84 | 89 | 94 | 98 |
| coated tablet 1-7 | 20 | 40 | 59 | 76 | 92 | 98 | 99 | 99 |
| coated tablet 1-8 | 20 | 30 | 45 | 57 | 69 | 80 | 90 | 98 |
| coated tablet 1-9 | 20 | 30 | 40 | 51 | 61 | 70 | 79 | 88 |
| coated tablet 1-10 | 20 | 26 | 36 | 44 | 50 | 57 | 64 | 66 |

From the data in Table 4, it can be seen that the coated tablets prepared with different compositions had wide drug release ranges. The release amount in 15 min was the content of the active ingredient Tofacitinib in the immediate release layer. The results showed that the coatings with different compositions released the active ingredient Tofacitinib within 15 min completely. In the subsequent process, the solid formulations can also continuously release the active ingredient Tofacitinib at a steady rate, providing effective and stable blood concentrations for a longer time. In particular, the release of formulations 1-1 to 1-8 was completed within 7 h, showing a more advantageous release effect.

### Example 3 Preparation of Osmotic Pump Tablets

1. Preparation:

Preparation was conducted according to the specific compositions and amounts (parts by weight, hereafter) in Table 5.

Tablet core granule: According to the metering ratio, Tofacitinib, the filler, the sustained release matrix material and half amount of lubricant were mixed uniformly using a hopper mixer (Nantong BEITE HBD-200); the above mixture was prepared into granules using a dry granulator (Alexanderwerk WP120, Germany), which were mixed with the remaining half amount of lubricant using a hopper mixer (Nantong BEITE HBD-200) to give the blended granules.

Compression: The above blended granules were pressed into tablet cores using a tableting machine (FETTE P2020).

Coating: Tofacitinib and membrane material were mixed uniformly, then added to an isopropanol solution containing 10 wt % purified water with sufficiently and uniformly stirring and prepared to be a suspension with a solid content of about 7%. The above suspension was sprayed onto tablet cores using a coating pan (O'Hara Lab coat M), which were dried to give semipermeable membrane coated tablets. During the spraying process, the material temperature was 25-35° C., and the inlet air volume was maintained as 50-100 cm³/h. After the spraying was completed, the semipermeable membrane coated tablets of Tofacitinib were dried in a 30° C. coating machine for 2 h, until the loss on drying was less than 2.0%.

Punching: The above semipermeable membrane coated tablets were punched by a mechanical puncher (BEKING Surom) with a 1.0 mm drill bit to give the final osmotic pump tablets.

According to the above procedure, the osmotic pump tablets 1-1 to 1-10 of the present disclosure were prepared.

TABLE 5
Specific composition of osmotic pump tablets (parts by weight)

| | | osmotic | osmotic | osmotic | osmotic | osmotic | osmotic | osmotic | osmotic | osmotic | osmotic |
|---|---|---|---|---|---|---|---|---|---|---|---|
| | | pump | pump | pump | pump | pump | pump | pump | pump | pump | pump |
| | | tablet | tablet | tablet | tablet | tablet | tablet | tablet | tablet | tablet | tablet |
| Component | Funcntion | 1-1 | 1-2 | 1-3 | 1-4 | 1-5 | 1-6 | 1-7 | 1-8 | 1-9 | 1-10 |

Sustained release tablet core

| | | | | | | | | | | | |
|---|---|---|---|---|---|---|---|---|---|---|---|
| Tofacitinib | Active | 9.5 | 9 | 8 | 7 | 6 | 5 | 8 | 8 | 8 | 8 |
| citrate | Ingredient | | | | | | | | | | |
| microcrystalline | filler | 93.2 | 93.5 | 94 | 94.5 | 95 | 95.5 | 114 | 94 | 74 | 34 |
| cellulose | | | | | | | | | | | |
| lactose | filler | 93.3 | 93.5 | 94 | 94.5 | 95 | 95.5 | 114 | 94 | 74 | 34 |
| hydroxyethyl | sustained | 200 | 200 | 200 | 200 | 200 | 200 | 160 | 200 | 240 | 320 |
| cellulose | release | | | | | | | | | | |
| | matrixmaterial | | | | | | | | | | |
| magnesium | lubricant | 4 | 4 | 4 | 4 | 4 | 4 | 4 | 4 | 4 | 4 |
| stearate | | | | | | | | | | | |

Coating prescription

| | | | | | | | | | | | |
|---|---|---|---|---|---|---|---|---|---|---|---|
| Tofacitinib | Active | 0.5 | 1 | 2 | 3 | 4 | 5 | 2 | 2 | 2 | 2 |
| citrate | Ingredient | | | | | | | | | | |
| cellulose | membrane | 10 | 10 | 10 | 10 | 10 | 10 | 4 | 20 | 30 | 40 |
| acetate | material | | | | | | | | | | |
| mannitol | filler | 5 | 5 | 5 | 5 | 5 | 5 | 2 | 10 | 15 | 20 |
| 90% | solvent | appro- | appro- | appro- | appro- | appro- | appro- | appro- | appro- | appro- | appro- |
| isopropanol* | | priate | priate | priate | priate | priate | priate | priate | priate | priate | priate |
| | | amount | amount | amount | amount | amount | amount | amount | amount | amount | amount |

*volatilized during preparation.

2. Dissolution Assay of the Formulations

According to the procedure described above, the dissolutions of each osmotic pump tablets in phosphate buffer, pH 6.8 were determined, and the results were shown in Table 6.

TABLE 6
Dissolutions of osmotic pump tablets

| | Dissolution (%) | | | | | | | |
|---|---|---|---|---|---|---|---|---|
| Time point (h) | 0.25 | 1 | 2 | 3 | 4 | 5 | 6 | 7 |
| osmotic pump tablet 1-1 | 5 | 9 | 40 | 66 | 84 | 89 | 97 | 97 |
| osmotic pump tablet 1-2 | 10 | 14 | 43 | 68 | 85 | 90 | 97 | 97 |
| osmotic pump tablet 1-3 | 20 | 22 | 49 | 72 | 87 | 91 | 97 | 97 |
| osmotic pump tablet 1-4 | 30 | 32 | 56 | 75 | 88 | 92 | 98 | 98 |
| osmotic pump tablet 1-5 | 40 | 43 | 62 | 79 | 90 | 93 | 99 | 99 |
| osmotic pump tablet 1-6 | 50 | 52 | 68 | 82 | 92 | 94 | 99 | 99 |
| osmotic pump tablet 1-7 | 20 | 55 | 78 | 93 | 99 | 99 | 99 | 99 |
| osmotic pump tablet 1-8 | 20 | 20 | 44 | 67 | 82 | 91 | 97 | 99 |
| osmotic pump tablet 1-9 | 20 | 20 | 37 | 52 | 65 | 75 | 80 | 82 |
| osmotic pump tablet 1-10 | 20 | 20 | 32 | 40 | 45 | 46 | 47 | 47 |

From the data in Table 6, it can be seen that the osmotic pump tablet prepared with different compositions had wide drug release ranges. The release amount in 15 min (0.25 h) was the content of the active ingredient Tofacitinib in the semipermeable membrane coating. The results showed that the coatings with different compositions released the active ingredient Tofacitinib within 15 min completely. In the subsequent process, the solid formulations can also continuously release the active ingredient Tofacitinib at a steady rate, providing effective and stable blood concentrations for a longer time. In particular, the release of formulations 1-1 to 1-8 was completed within 7 h, showing a more advantageous release effect.

### Example 4 Comparison of Dissolutions

According to the above procedure, the dissolutions of Tofacitinib formulations among different dosage forms (20% for the immediate release portion of Tofacitinib and 80% for the sustained release portion of Tofacitinib) were determined for comparison, where the dissolution of each formulation was that in phosphate buffer, pH 6.8. The results were shown in Table 7 below.

TABLE 7
Dissolutions of formulations

| | Dissolution (%) | | | | | | | |
|---|---|---|---|---|---|---|---|---|
| Time point (h) | 0.25 | 1 | 2 | 3 | 4 | 5 | 6 | 7 |
| bilayer tablet 1-3 | 20 | 39 | 54 | 65 | 74 | 83 | 91 | 97 |
| coated tablet 1-3 | 20 | 33 | 49 | 62 | 74 | 83 | 91 | 97 |
| osmotic pump tablet 1-3 | 20 | 22 | 49 | 72 | 87 | 91 | 97 | 97 |

From the data in the table above, it can be seen that the above three different dosage forms of Tofacitinib formulations all have a special release pattern of immediate release in the first 15 min and then sustained release for more than 5 h. Such release pattern not only maintains the rapid onset of action after administration in the early stage, but also maintains the clinical effect for a long time, providing more stable blood concentrations.

### Example 5 Pharmacokinetic Studies

The coated tablets 1-3 prepared in Example 2 were used.

1. Testing Formulations

Testing formulation T: Tofacitinib coated tablets were prepared according to the compositions and procedure of coated tablets 1-3 in Example 2, with the strength of 11 mg, once a day.

Reference formulation R: commercial Tofacitinib sustained release tablet (Xeljanz XR), with the strength of 11 mg, once a day.

2. Testing Procedure

1) Single Administration Test

An open, randomized, double-period, double-crossover self-controlled trial design method (7-day washout period) was used, where 10 healthy male subjects were randomly divided into 2 groups where 5 in each group. The grouping design was shown in the table below:

TABLE 8
Grouping design

| Period | Group 1 | Group 2 |
|---|---|---|
| Period I | T | R |
| Period II | R | T |

The subjects entered the Phase I clinical trial ward before the testing day, were given a uniform light diet in the evening, and then fasted overnight (at least 10 h, free for getting water). The subjects were orally given 11 mg of Tofacitinib coated tablets with empty stomach at about 8 am the next day and were given 240 ml of water after 10 min. Alternatively, the subjects were orally given 11 mg of Tofacitinib sustained release tablets with empty stomach at about 8 am and 8 pm the next day, respectively along with 240 ml of water. No water was allowed for 2 h after administration with the upper body upright. The lunches were given after 4 h.

Blood sample collection procedure: 0 h before administration (within 1 h before administration) and 0.5, 1, 1.5, 2, 2.5, 3, 3.5, 4, 4.5, 5, 6, 7, 8, 12, 24, 36 h after administration (17 blood collection points), 4 mL of cubital venous blood was collected, and the blood samples were placed in blood collection tubes anticoagulated with heparin sodium. After collection, the samples were centrifuged at 3500 rpm for 5 min at 4° C. The plasma was divided into two parts, 1 mL of plasma was added to the formal tube and the remaining plasma was added to the backup tube. The samples were stored in a −20° C. refrigerator 2 h after collection for subsequent use.

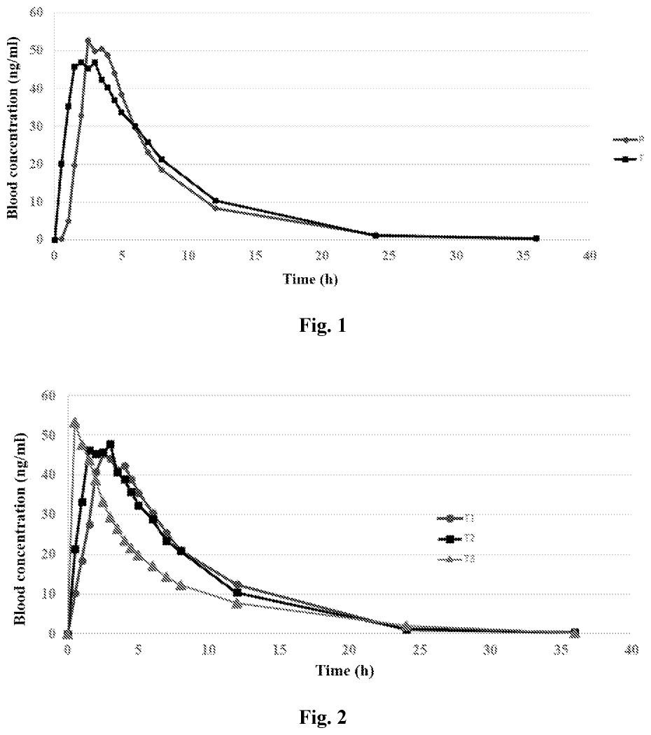

The LC-MS/MS method was used to determine the concentration of Tofacitinib in each plasma sample. The pharmacokinetic statistical software DAS 2.0 was used for calculation and biostatistical analysis was performed. The test results were shown in Table 9 and FIG. 1.

From the blood concentration curve, it can be seen that the time for the reference sustained release tablet to reach 17 ng/ml was about 90 min. In contrast, the time for the coated tablet of the present disclosure to reach 17 mg/ml was about 30 min.

TABLE 9
Main pharmacokinetic parameters of Tofacitinib single administration

| | Tofacitinib sustained | Tofacitinib |
|---|---|---|
| | release tablet | coated tablet |
| | Commercial (R) | (T) |
| Dosage form | Sustained release | Coated tablet |
| | tablet | (Sustained release) |
| Administration Route | oral | oral |
| Dosage | 11 mg, Once a day | 11 mg, Once a day |
| Cₘₐₓ (ng/mL) | 65 ± 28 | 57 ± 25 |
| AUC₀₋₃₆ₕ (ng · h/ml) | 367 ± 116 | 414 ± 120 |
| Tₘₐₓ (h) | 3.0[2.5~4.0] | 2.1[1.2~3.4] |
| Ta (h) | 7.6 | 10.1 |

Ta refers to the time that the blood concentration can be maintained above 17 ng/mL (IC₅₀).

It can be seen from Table 9 that the maximum blood concentration Cₘₐₓ of the Tofacitinib coated tablet of the present disclosure was significantly lower than that of the commercially available sustained release tablet, where the fluctuation was more stable than that of the commercially available sustained release tablet, and the area under the curve of blood concentration was significantly increased. The Ta value was extended by about 33% as compared to that of Tofacitinib commercial sustained release tablet.

### Example 6 Pharmacokinetic Studies of Tofacitinib Formulation

The coated tablets 1-2, 1-3 and 1-6 prepared in Example 2 were used.

1. Testing Formulations

Testing formulation T1: Tofacitinib coated tablet prepared according to the composition and procedure of coated tablet 1-2 in Example 2 with strength of 11 mg, once a day.

Testing formulation T2: Tofacitinib coated tablet prepared according to the composition and procedure of coated tablet 1-3 in Example 2 with strength of 11 mg, once a day.

Testing formulation T3: Tofacitinib coated tablet prepared according to the composition and procedure of coated tablet 1-6 in Example 2 with strength of 11 mg, once a day.

2. Testing Procedure

1) Single Administration Test

An open, randomized, double-period, triple-crossover self-controlled trial design method (7-day washout period) was used, where 9 healthy male subjects were randomly divided into 3 groups where 3 in each group. The grouping design was shown in the table below:

TABLE 10
Grouping design

| Period | Group 1 | Group 2 | Group 3 |
|---|---|---|---|
| Period I | T1 | T3 | T2 |
| Period II | T2 | T1 | T3 |
| Period III | T3 | T2 | T1 |

The subjects entered the Phase I clinical trial ward before the testing day, were given a uniform light diet in the evening, and then fasted overnight (at least 10 h, free for getting water). The subjects were orally given 11 mg of Tofacitinib coated tablets with empty stomach at about 8 am the next day and were given 240 ml of water after 10 min. No water was allowed for 2 h after administration with the upper body upright. The lunches were given after 4 h.

Blood sample collection procedure: 0 h before administration (within 1 h before administration) and 0.5, 1, 1.5, 2, 2.5, 3, 3.5, 4, 4.5, 5, 6, 7, 8, 12, 24, 36 h after administration (17 blood collection points), 4 mL of cubital venous blood was collected, and the blood samples were placed in blood collection tubes anticoagulated with heparin sodium. After collection, the samples were centrifuged at 3500 rpm for 5 min at 4° C. The plasma was divided into two parts, 1 mL of plasma was added to the formal tube and the remaining plasma was added to the backup tube. The samples were stored in a −20° C. refrigerator 2 h after collection for subsequent use.

The LC-MS/MS method was used to determine the concentration of Tofacitinib in each plasma sample. The pharmacokinetic statistical software DAS 2.0 was used for calculation and biostatistical analysis was performed. The test results were shown in Table 11 and FIG. 2.

TABLE 11
Main pharmacokinetic parameters of Tofacitinib single administration

| | Tofacitinib | Tofacitinib | Tofacitinib |
|---|---|---|---|
| | coated tablet | coated tablet | coated tablet |
| | (T1) | (T2) | (T3) |
| Dosage form | coated tablet | coated tablet | coated tablet |
| Administration Route | oral | oral | oral |
| Dosage | 11 mg, | 11 mg, | 11 mg, |
| | Once a day | Once a day | Once a day |
| Cₘₐₓ (ng/mL) | 50 ± 23 | 58 ± 22 | 70 ± 32 |
| AUC₀₋₃₆ₕ (ng · h/ml) | 404 ± 140 | 406 ± 123 | 324 ± 111 |
| Tₘₐₓ (h) | 2.9[2.0~3.7] | 2.4[1.2~3.8] | 0.7[0.3~1.0] |
| Ta (h) | 8.6 | 9.7 | 5.8 |

Ta refers to the time that the blood concentration can be maintained above 17 ng/mL (IC₅₀).

From the blood concentration curves, it can be seen that the time for the testing formulations T1, T2 and T3 to reach the blood concentration of 17 ng/ml were about 50 min, 25 min and 12 min respectively.

It can be seen from Table 11 that the blood concentrations of the formulations of the present disclosure showed smooth fluctuation, and higher area under the curve of the blood concentration. The Ta values of the testing formulations T1, T2 and T3 were 8.6, 9.7 and 5.8 h, respectively. It can be seen that the formulations of the present disclosure can achieve high Ta value, showing better therapeutic effects. In particular, testing formulations T1, T2 can achieve significantly higher Ta values and higher area under the curve of blood concentration, showing a particular advantage.

The preferable embodiments of the present disclosure are provided above and it should be understood that those skilled in the art, without departing from the principles of the present disclosure, can make changes and modifications, and such changes and modifications should also be encompassed with the scope of the present disclosure.

1. A solid formulation, wherein the solid formulation comprises an immediate release portion and a sustained release portion, and comprises active ingredients, wherein the weight ratio of the active ingredient in the sustained release portion to the active ingredient in the immediate release portion is about 1:1 or higher.

2. The solid formulation according to claim 1, wherein
the weight ratio of the active ingredient in the sustained release portion to the active ingredient in the immediate release portion is selected from the group consisting of about 1:1-20:1, about 1:1-9:1, 1:1-4:1, about 1:1-7:3, about 4:1-9:1 and about 7:3-4:1.

3. The solid formulation according to claim 1, wherein
the active ingredient is Tofacitinib or a pharmaceutically acceptable salt thereof.

4. The solid formulation according to claim 1, wherein
the sustained release portion comprises a sustained release matrix material.

5. The solid formulation according to claims 1-4, wherein
the sustained release portion and/or the immediate release portion comprises a film-forming material.

6. The solid formulation according to claim 1, wherein
the active ingredient in the solid formulation has a total dissolution within 15 min selected from the group consisting of about 5-50%, about 10-30%, and about 20%;
the active ingredient in the solid formulation has a total dissolution within 1 h selected from the group consisting of about 20-60%, and about 30-50%;
the active ingredient in the solid formulation has a total dissolution within 3 h selected from the group consisting of about 55-95%, and about 60-80%; and/or
the active ingredient in the solid formulation has a total dissolution within 5 h selected from the group consisting of at least about 80%, and at least about 85%.

7. The solid formulation according to claim 1, wherein
after a single administration, the time require by the solid formulation for achieving a blood concentration of 17 ng/ml is selected from the group consisting of about 10-60 min, about 25-45 min, and about 20-30 min; and/or
after a single administration, the duration time of the solid formulation for achieving a blood concentration over 17 ng/ml is selected from the group consisting of about 8-12 h, and about 9-11 h.

8. The solid formulation according to claim 1, which is in the form of a tablet, a granule, a pellet or a capsule.

9. The solid formulation according to claim 1, wherein
the immediate release portion and the sustained release portion each independently comprise one or more pharmaceutically excipients selected from the group consisting of a sustained release matrix material, a film-forming material, a binder, a filler, a disintegrant, and a lubricant.

10. The solid formulation according to claim 1, wherein
the solid formulation is a bilayer tablet, and comprises the active ingredient, the filler, the disintegrant, the lubricant and the sustained release matrix material; or
the solid formulation is a coated tablet, and comprises the active ingredient, the filler, the sustained release matrix material, the lubricant, and the film-forming material; or
the solid formulation is an osmotic pump tablet, and comprises the active ingredient, the filler, the sustained release matrix material, the lubricant, and the film-forming material; or
the solid formulation is a capsule, and comprises the active ingredient, the filler, the binder, the lubricant, and the film-forming material.

11. The solid formulation according to claim 1, wherein
the sustained release portion comprises the film-forming material, and
the active ingredient of the immediate release portion is within the film-forming material.

12. A pharmaceutical composition, comprising the solid formulation according to claim 1.

13. A method for treating or preventing a disease, comprising administering the solid formulation according to claim 1 to a subject in need thereof, wherein the the manufacture of a medicament for treating or preventing a disease comprising lupus, multiple sclerosis, rheumatoid arthritis, psoriatic arthritis, type I diabetes and diabetic complications, cancer, asthma, atopic dermatitis, autoimmune thyroid disease, moderate-to-severe active ulcerative colitis, Crohns' disease, ankylosing spondylitis, juvenile idiopathic arthritis, immune system disorder baldness, vitiligo, systemic lupus erythematosus, Alzheimer's disease or leukemia.

14. A process for preparing the solid formulation according to claim 1, comprising
1) combining the active ingredient with one or more pharmaceutically acceptable excipients to give the immediate release portion,
2) combining the active ingredient with one or more pharmaceutically acceptable excipients to give the sustained release portion, and
3) combing the immediate release portion and the sustained release portion to give the solid formulation;
wherein
step 1) and step 2) are conducted in any order independently; and
the active ingredients and the excipients in step 1) and step 2) are used independently.

15. The solid formulation according to claim 1, wherein
the active ingredient is Tofacitinib citrate.

16. The solid formulation according to claim 4, wherein
the sustained release matrix material is selected from the group consisting of about 40%-80%, about 40%-50% and about 50%-70% of the weight of the sustained release portion.

17. The solid formulation according to claim 5, wherein
the film-forming material is selected from the group consisted of about 30%-70% and about 40%-60% of the weight of the sustained release portion; and/or
the film-forming material is selected from the group consisted of about 40%-80% and about 50%-70% of the weight of the immediate release portion.

18. The solid formulation according to claim 8, wherein
the tablet is a coated tablet or an osmotic pump tablet, and the coated tablet or the osmotic pump tablet optionally comprises a sustained coating.

19. The solid formulation according to claim 9, wherein
the sustained release matrix material is one or more selected from the group consisting of: hydroxypropyl methyl cellulose, hydroxypropyl cellulose, hydroxyethyl cellulose, polyoxyethylene, glyceryl behenate, polymethylmethacrylate, acrylic resin, povidone, xanthan gum, sodium alginate, calcium alginate, carbomer, chitin, polyethylene, and polysiloxane;
the film-forming material is one or more selected from the group consisting of: cellulose acetate, ethyl cellulose, hydroxypropyl methyl cellulose, and acrylic resin;
the binder is one or more selected from the group consisting of: polyethylene glycol, starch, hydroxypropyl cellulose, hydroxypropyl methyl cellulose, pregelatinized starch, polyvinylpyrrolidone, gum arabic, and gelatin;
the filler is one or more selected from the group consisting of: starch, pregelatinized starch, modified starch, microcrystalline cellulose, silicified microcrystalline cellulose, low-substituted hydroxypropyl cellulose glucose, sucrose, lactose, sorbitol, mannitol, erythritol, calcium carbonate and calcium hydrophosphate;
the disintegrant is one or more selected from the group consisting of: microcrystalline cellulose, carboxymethyl cellulose, copovidone, croscarmellose sodium, calcium carboxymethylcellulose, sodium carboxymethyl starch, low-substituted hydroxypropyl cellulose, hydroxymethyl starch, alginic acid, sodium alginate, guar gum, corn starch and magnesium aluminum silicate;
the lubricant is one or more selected from the group consisting of: magnesium stearate, stearic acid, stearate, sodium stearyl fumarate, sodium lauryl sulfate, polyethylene glycol, sodium benzoate, sucrose fatty acid ester, micronized silica gel, talcum powder, glyceryl monostearate, glyceryl behenate, glyceryl palmitostearate, stearic acid, and hydrogenated vegetable oil.

20. A method for treating or preventing a disease, comprising administering the pharmaceutical composition according to claim 12 to a subject in need thereof, wherein the disease comprising lupus, multiple sclerosis, rheumatoid arthritis, psoriatic arthritis, type I diabetes and diabetic complications, cancer, asthma, atopic dermatitis, autoimmune thyroid disease, moderate-to-severe active ulcerative colitis, Crohns' disease, ankylosing spondylitis, juvenile idiopathic arthritis, immune system disorder baldness, vitiligo, systemic lupus erythematosus, Alzheimer's disease or leukemia.
